(19) United States
(12) Patent Application Publication
(10) Pub. No.: US 2020/0216335 A1
(43) Pub. Date: Jul. 9, 2020

(54) PORTABLE APPARATUS FOR CREATING MIST USING STERILIZED WATER

(71) Applicant: SOOVON Co., Ltd., Incheon (KR)

(72) Inventor: Kyung Hee OH, Goyang-si (KR)

(73) Assignee: SOOVON Co., Ltd., Incheon (KR)

(21) Appl. No.: 16/523,356

(22) Filed: Jul. 26, 2019

(30) Foreign Application Priority Data
Jan. 9, 2019 (KR) 10-2019-0002577

(51) Int. Cl. C02F 1/46; A61Q 19/00; A61K 8/19; A61K 8/04

(52) U.S. Cl. CPC C02F 1/4608; A61Q 19/00; C02F 2103/026; A61K 8/046; C02F 2303/04; A61K 8/19

(57) ABSTRACT

Disclosed herein is a portable mist device which can spray sterilized hydrogen water in a mist form through an ultrasonic vibrator, thereby aiding beauty care. The portable mist device includes an upper container having a raw water inlet, a lower container having a sterilized water outlet formed at the side, and a pole plate unit for generating sterilized water through underwater discharge of the raw water by a pair of electrode plates. The electrode plates of the pole plate unit are mounted vertically and are located parallel with the sterilized water outlet. Therefore, the portable mist device can enhance mist discharge efficiency, and be easily connected with a battery arranged at a lower portion without the need to bend electrode connection terminals extending from one end of each electrode plate. So, the portable mist device can reduce costs required for bending the electrode connection terminals and facilitate production of standardized products.

### BACKGROUND OF THE INVENTION

### Field of the Invention

The present invention relates to a portable device for providing sterilized hydrogen water in a mist form, and more particularly, to a portable device which can spray sterilized hydrogen water in a mist form through an ultrasonic vibrator, thereby aiding beauty care.

### Background Art

Recently, not only women but also men pay attention to beauty care in order to maintain beautiful looks. In general, cosmetics ingredients, such as collagen, nutritive emulsion, essence, and others, have been widely used in order to help moisture preservation, nutrition supply and activation of skin functions for beauty care.

Moreover, a mist supplier has been used for skin care in order to spray liquefied cosmetics in a fine-grained mist form.

The mist supplier may be used for various purposes, such as moisture preservation, whitening effect, nutrition supply, and so on, and has been widely used for moisture preservation to prevent a dry skin.

The mist supplier is divided into a manual spray type mist supplier, a stationary steam heating type mist supplier.

However, the conventional manual spray type mist supplier has an inconvenience in that a user has to press a button every time the user uses the mist sprayer. Furthermore, the conventional steam heating type mist supplier has a merit in that it is possible to spray liquid in a relatively small and uniform particle form since atomizing and spraying liquid in a steam type by heating the liquid, but has a disadvantage in that the size of the mist supplier is large and it is impossible to carry since it requires relative large electric power to operate a heating device.

In order to solve the above problems, Korean Patent Publication No. 10-2010-0057339 discloses a portable moisture supplier using an ultrasonic vibrator, and Korean Patent Publication No. 10-2014-0123796 discloses a mist supplier capable of automatically spraying mist in a slidably opening and closing manner.

Additionally, Korean Patent No. 10-1747913 discloses a portable mist device capable of sterilizing liquid to be supplied to the skin for a user's hygiene and safety.

The portable moisture supplier disclosed in Korean Patent Publication No. 10-2010-0057339 and the portable mist device disclosed in Korean Patent Publication No. 10-2014-0123796 supply the existing liquid cosmetics or water in a mist form, but the portable mist device disclosed in Korean Patent No. 10-1747913 sterilizes and treats the liquid supplied to the skin in order to reinforce the user's hygiene and safety.

However, the portable mist device disclosed in Korean Patent No. 10-1747913 has several disadvantages in that it is restricted in space utilization since a pole plate unit for generating sterilized water through underwater discharge of raw water is arranged horizontally, and in that mist discharge efficiency is reduced since an electrode plate for generating sterilized water is located at right angles to a sterilized water outlet.

In addition, the portable mist device has further disadvantages in that it is inconvenient to arrange electrode connection terminals and connect the electrode connection terminals with a power source, it takes much time and manpower to bend the electrode connection terminals, and bending angles are not equal since a user has to bend and treat the electrode connection terminals one by one.

### PATENT LITERATURE

### Patent Documents

Patent Document 1: Korean Patent Publication No. 10-2010-0057339

Patent Document 2: Korean Patent Publication No. 10-2014-0123796

Patent Document 3: Korean Patent No. 10-1747913

### SUMMARY OF THE INVENTION

Accordingly, the present invention has been made to solve the above-mentioned problems occurring in the prior arts, and it is an object of the present invention to provide a portable mist device for providing sterilized hydrogen water mist capable of providing sterilized hydrogen water in a mist form using an ultrasonic vibrator and using an inner space more effectively and enhancing mist discharge efficiency by arranging an electrode plate vertically.

To accomplish the above object, according to the present invention, there is provided a portable mist device for providing sterilized hydrogen water mist including: a sterilized water generating module, which includes an upper container having a raw water inlet, a lower container having a sterilized water outlet formed at the side, and a pole plate unit for generating sterilized water through underwater discharge of the raw water by a pair of electrode plates; a lid housing disposed to be combined with the raw water inlet; a spraying unit disposed at the sterilized water outlet to discharge the sterilized water in a mist form to the outside through an ultrasonic vibrator; and a power supply unit for supplying electric power to the pole plate unit and the ultrasonic vibrator, wherein the lower container has pole plate unit mounting grooves to detachably mount the pole plate unit, the electrode plates are vertically mounted in the pole plate unit mounting grooves and the pole plate unit mounting grooves are located parallel with the sterilized water outlet.

The portable mist device for providing sterilized hydrogen water mist further includes an upper housing for surrounding the upper container; and a middle and lower housing for surrounding the lower container and the power supply unit.

The pole plate unit includes a pair of electrode plates and a partition plate arranged between the electrode plates. In this instance, the pole plate unit mounting grooves are formed in such a way that the pole plate unit is detachably mounted. That is, one or more pole plate units may be inserted into one or more pole plate unit mounting grooves. In other words, if there are two or more pole plate units, two or more pole plate unit mounting grooves are provided.

The lower container has two pole plate unit mounting grooves in which a pair of the electrode plates are individually attached and detached. In this instance, a partition plate is formed between the two grooves to prevent physical contact of the two electrode plates.

Each of the electrode plates has an electrode connection terminal extending and protruding in a vertical direction to a predetermined length from a lower end thereof.

The lower container has an electrode connection terminal through hole formed at a lower end thereof so that the electrode connection terminal of each electrode plate mounted through the pole plate unit mounting grooves pass therethrough.

The lid housing has a gas discharge hole for discharging gas to the outside, and the gas discharge hole is stopped with a member made of a soft material.

The gas discharge hole stopping member is cut in a predetermined shape to be opened and closed depending on gas pressure.

The lid housing has a magnet receiving recess to receive a magnet. In this instance, a cover for covering the lid housing has a massage protrusion formed at a position corresponding to the magnet receiving recess to provide massage effect.

According to the present invention, the portable mist device for providing sterilized hydrogen water mist can generate sterilized hydrogen and provide the sterilized hydrogen water in a mist form through an ultrasonic vibrator. Especially, the pole plate unit for generating sterilized water through underwater discharge of raw water is mounted vertically and is arranged parallel with the sterilized water outlet.

Therefore, the portable mist device for providing sterilized hydrogen water mist can use the inner space more effectively and enhance mist discharge efficiency.

Moreover, when the pole plate unit is mounted vertically, the electrode connection terminals extending at one end of the electrode plate can be easily connected with the battery arranged below without bending.

Therefore, the portable mist device for providing sterilized hydrogen water mist can reduce the costs required for bending the electrode connection terminals and facilitate production of standardized products.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIG. 1 is a view showing a portable mist device for providing sterilized hydrogen water mist according to the present invention;

FIG. 2 is a view showing an outward appearance of the portable mist device for providing sterilized hydrogen water mist according to the present invention;

FIG. 3 is a view showing an internal configuration of the portable mist device for providing sterilized hydrogen water mist according to the present invention;

FIG. 4 is a view showing an upper container and a lower container of a sterilized water generating module;

FIG. 5 is a view showing a pole plate unit;

FIG. 6 is a view showing an example of pole plate unit mounting grooves disposed in the lower container;

FIG. 7 is a view showing another example of the pole plate unit mounting grooves disposed in the lower container;

FIG. 8 is a view showing a through hole of an electrode connection terminal;

FIG. 9 is a view showing a state where the electrode connection terminal is mounted in the lower container;

FIG. 10 is a view showing a gas discharge through a lid housing; and

FIG. 11 is a view showing a magnet receiving hole and a massage protrusion formed on the lid housing and a cover.

### DETAILED DESCRIPTION OF THE PREFERRED EMBODIMENT

The example embodiments of the present invention are capable of various modifications and alternative forms, and particular embodiments of the present invention will be illustrated in the attached drawings and described in this specification in detail. It should be understood, however, that there is no intent to limit example embodiments of the invention to the particular forms disclosed, but on the contrary, example embodiments of the invention are to cover all modifications, equivalents, and alternatives falling within the technical idea and scope of the present invention.

In the following description of the present invention, a detailed description of known functions and configurations incorporated herein will be omitted when it may make the subject matter of the present invention rather unclear.

It will be further understood that the words or terms used in the present invention are used to describe specific embodiments of the present invention and there is no intent to limit the present invention. The singular form of the components may be understood into the plural form unless otherwise specifically stated in the context.

It should be also understood that the terms of ‘include’ or ‘have’ in the specification are used to mean that there are characteristics, numbers, steps, operations, components, parts, or combinations of the steps, operations, components and parts described in the specification and there is no intent to exclude existence or possibility of other characteristics, numbers, steps, operations, components, parts, or combinations of the steps, operations, components and parts.

It will be understood that terms, such as “first” or “second” may be used in the specification to describe various components but are not restricted to the above terms. The terms may be used to discriminate one component from another component.

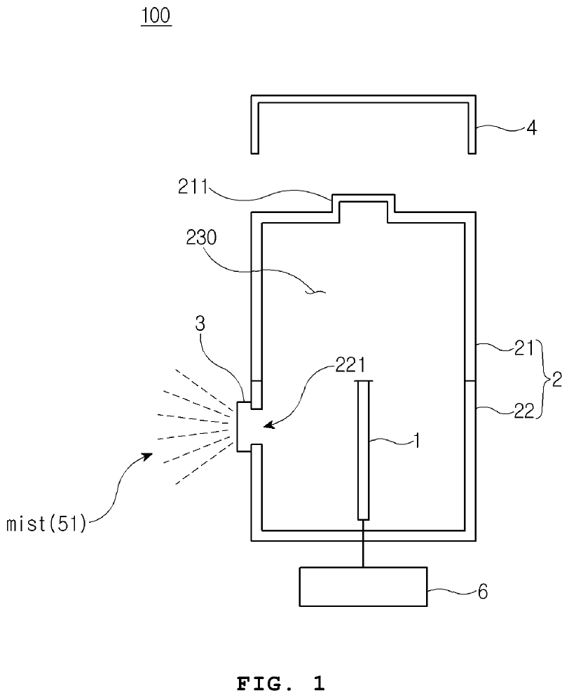

Referring to FIG. 1, a portable mist device 100 for providing sterilized hydrogen water mist is to provide sterilized hydrogen water (hydrogen water and/or sterilized water) used for beauty care in a mist form, and includes a sterilized water generating module 2, a spraying unit 3, a lid housing 4, and a power supply unit 6.

The sterilized water generating module 2 includes an upper container 21 having a raw water inlet 211, a lower container 22 having a sterilized water outlet 221 formed at the side, and a pole plate unit 1 for generating sterilized water through underwater discharge of raw water by a pair of electrode plates.

The upper container 21 and the lower container 22 may be formed to be attached and detached, and may be varied in their forms, sizes, materials, and detachable structures. An inner space 230 formed by the upper container 21 and the lower container 22 which are combined with each other stores raw water supplied through the raw water inlet 211, and the pole plate unit 1 is arranged in the inner space 230.

The pole plate unit 1 makes the raw water stored in the inner space 230 into a plasma ion state through underwater discharge. Therefore, the pole plate unit 1 generates various anions, such as O—, O₃—, OH—, HOCl, H₂O₂, and others, to sterilize various germs, viruses, and bacteria in water.

The pole plate unit 1 is mounted vertically through pole plate unit mounting grooves disposed in the lower container 22. When the pole plate unit 1 is mounted, since sides of the electrode plates are located parallel with the side of the sterilized water outlet 221, sterilized water is supplied to the spraying unit 3 so as to enhance efficiency to discharge mist 51.

The lid housing 4 may be formed to be combined with the raw water inlet 211, and is located at an upper end of the upper container 21.

The spraying unit 3 discharges the sterilized water supplied through the sterilized water outlet 221 in the mist form to the outside using an ultrasonic vibrator. The sterilized water outlet 221 is located at the side of the lower container, and the spraying unit 3 discharges the mist 51 in a perpendicular direction to a central axis of the lower container 22.

The power supply unit 6 supplies electric power to the pole plate unit 1 and the spraying unit 3 and uses a battery. The power supply unit 6 may be arranged in various ways as occasion demands. For instance, the power supply unit 6 may be arranged below the lower container 22.

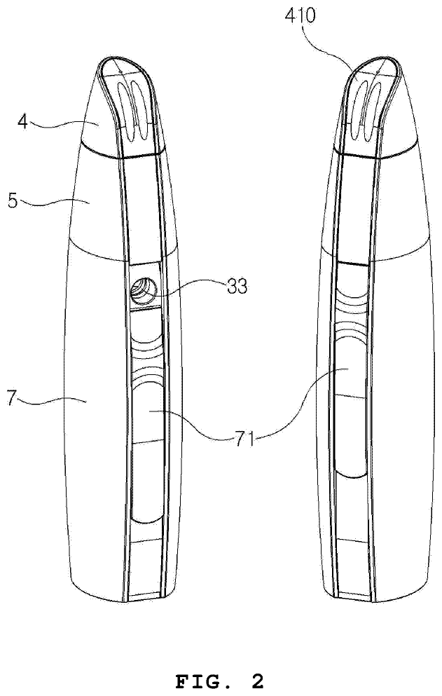

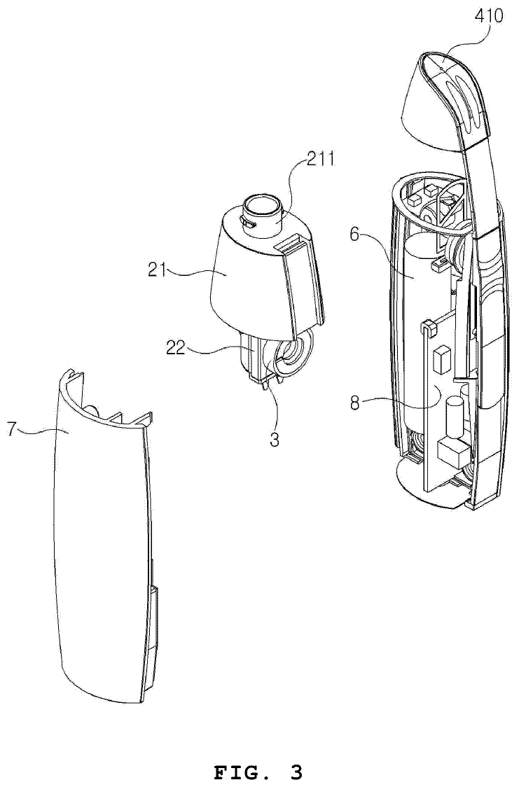

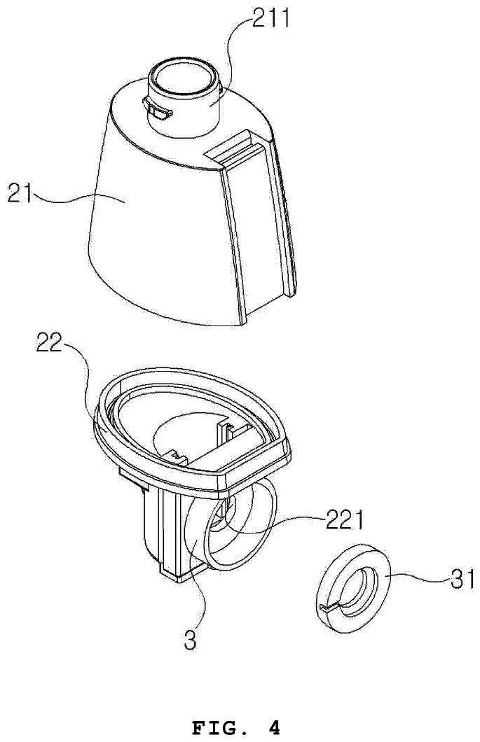

FIG. 2 illustrates a concrete example of the outward appearance of the portable mist device 100 for providing sterilized hydrogen water mist, FIG. 3 is an exploded perspective view showing an internal configuration of the mist device 100, and FIG. 4 is a view showing an example of the upper container 21 and the lower container 22.

As shown in FIG. 2, the portable mist device 100 for providing sterilized hydrogen water mist includes an upper housing 5 surrounding the upper container 21 and a middle and lower housing 7 surrounding the lower container 22 and the power supply unit 6.

The upper housing 5 surrounds the upper container 21 without any limitation in its form or material. Furthermore, the upper housing 5 may be integrated with the upper container 21 or the upper container 21 may perform the function of the upper housing 5 without forming the upper housing 5.

If the upper container 21 and the upper housing 5 are transparent, a user can check the degree and form of the sterilized water generated in the upper container 21 in real time and easily check an amount of the raw water stored in the upper container 21. So, the user can easily replace or refill water if necessary.

The middle and lower housing 7 may include a spraying unit opening and closing unit 71 for opening and closing the mist spray hole 33 through which the mist 51 is discharged. In this embodiment, the spraying unit opening and closing unit 71 interlocks with the power supply unit 6 to supply electric power to the pole plate unit 1 and the ultrasonic vibrator 31 when the mist spray hole 33 is opened. That is, the spraying unit opening and closing unit 71 is operated by a switch to actuate the pole plate unit 1 and the ultrasonic vibrator 31 when the spraying unit opening and closing unit 71 moves.

The portable mist device 100 for providing sterilized hydrogen water mist may further include a PCB module 8 on which an electric circuit is formed, a rubber packing for preventing water leak, and other various elements.

The sterilized water generating module 2 includes the upper container 21 and the lower container 22, and the pole plate unit 1 is detachably mounted inside the lower container 22.

The upper container 21 has the raw water inlet 211 which receives the raw water from the outside, the lower container 22 has the sterilized water outlet 221, and the pole plate unit 1 generates sterilized water through underwater discharge of the raw water.

The raw water inlet 211 may be located at the side or the upper surface of the upper container 21, but it is preferable to be located at the upper surface of the upper container 21 for vertical supply of the raw water.

If the portable mist device 100 for providing sterilized hydrogen water mist discharges sterilized water from the side, the sterilized water outlet 221 and the spraying unit 3 may be formed to face the side in a direction to be perpendicular to the central axis of the mist device 100.

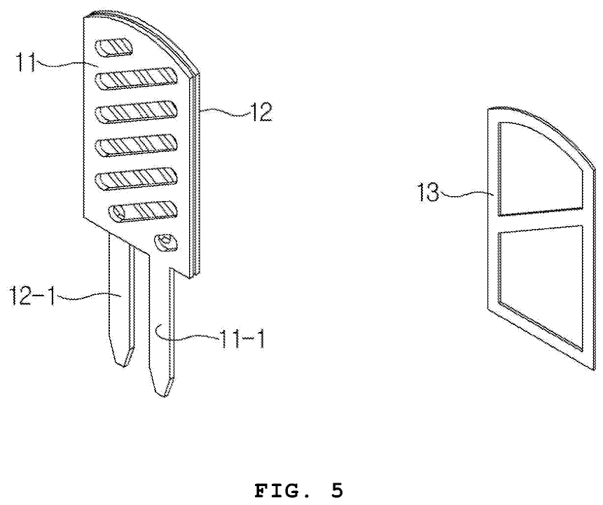

FIG. 5 is a view showing the pole plate unit 1. The pole plate unit 1 includes a pair of electrode plates 11 and 12 in order to generate sterilized water through underwater discharge of raw water.

FIG. 5 illustrates an example of the electrode plates 11 and 12 which are thin plates having the width longer than the height and have a plurality of diagonal grooves. The pole plate unit 1 may have one among various forms well-known in the related field.

FIG. 5 illustrates an example of a partition plate 13 having the same shape as the electrode plates, a hollow inner space, and a diagonal slot. The partition plate 13 is arranged between the electrode plates 11 and 12 to prevent physical contact of the electrode plates 11 and 12.

FIG. 5 illustrates an example to help understanding of the description, and the forms of the electrode plates 11 and 12 and the partition plate 13 may be varied if necessary.

In order to apply electric power with different poles to the electrode plates 11 and 12, the electrode plates 11 and 12 have electrode connection terminals 11-1 and 12-1 which extend and protrude from lower ends to a predetermined length in a vertical direction.

Because the pole plate unit 1 is vertically mounted through the pole plate unit mounting grooves formed inside the lower container 22, the electrode connection terminals 11-1 and 12-1 formed on the electrode plates 11 and 12 can be used as they are without the need to be bent.

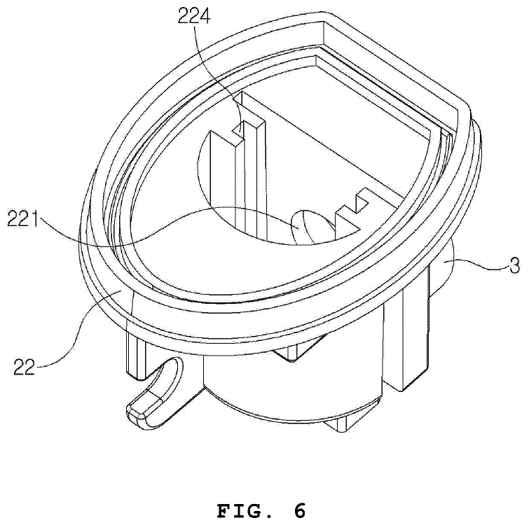

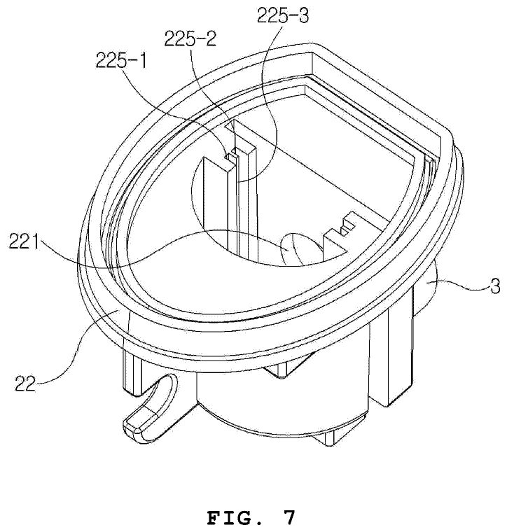

FIGS. 6 and 7 illustrate different examples of the pole plate unit mounting grooves disposed in the lower container 22.

FIG. 6 illustrates the pole plate unit mounting grooves 224 formed at both sides of the lower container 22 one by one. That is, the pole plate unit mounting grooves 224 are formed vertically on both sides inside the lower container 22.

In FIG. 6, the pole plate unit 1 is detachably mounted on the pole plate unit mounting grooves 225 in a state where the partition plate 13 is arranged between a pair of the electrode plates 11 and 12. That is, a pair of the electrode plates 11 and 12 and the partition plate 13 arranged between the electrode plates 11 and 12 are mounted to or detached from the pole plate unit mounting grooves 224.

FIG. 7 illustrates a state where two pole plate unit mounting grooves 225-1 and 225-2 are formed on each of both sides of the lower container 22. That is, the two pole plate unit mounting grooves 225-1 and 225-2 are formed vertically on each of both sides inside the lower container 22.

The two electrode plates 11 and 12 of the pole plate unit 1 are individually mounted on the pole plate unit mounting grooves 225-1 and 225-2, and a partition plate 225-3 is formed between the two pole plate unit mounting grooves 225-1 and 225-2 to prevent physical contact of the two electrode plates 11 and 12.

In this embodiment, because the electrode plates 11 and 12 are separated from each other physically, the partition plate 13 of the pole plate unit 1 is not needed.

As shown in FIGS. 6 and 7, the pole plate unit 1 is mounted by the pole plate unit mounting grooves formed inside the lower container 22, and there are various structures to make the pole plate unit 1 be mounted detachably.

Especially, the sterilized water outlet 221 may be formed to face the side of the portable mist device 100 for providing sterilized hydrogen water mist, and when the pole plate unit 1 is mounted, the pole plate unit mounting grooves are configured such that sides of the electrode plates 11 and 12 are located parallel with the side of the sterilized water outlet 221. For this, the pole plate unit mounting grooves 224 or 225-1 and 225-2 may be formed in the vertical direction to face the lower container 22 from the upper container 21.

As shown in FIG. 5a, the electrode connection terminals 11-1 and 12-1 are spaced apart from each other at a predetermined interval in vertical and horizontal directions.

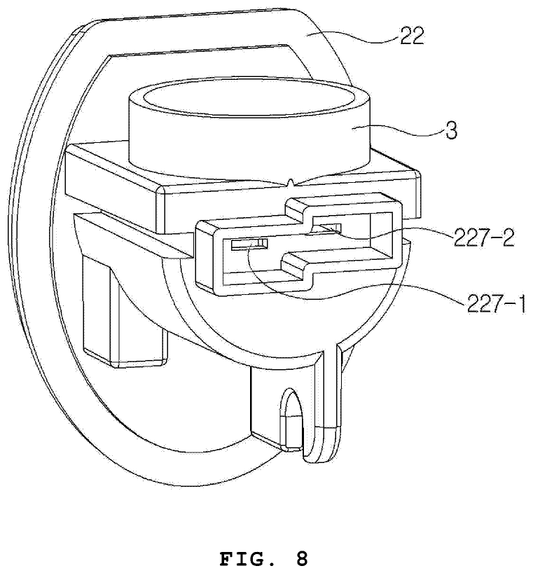

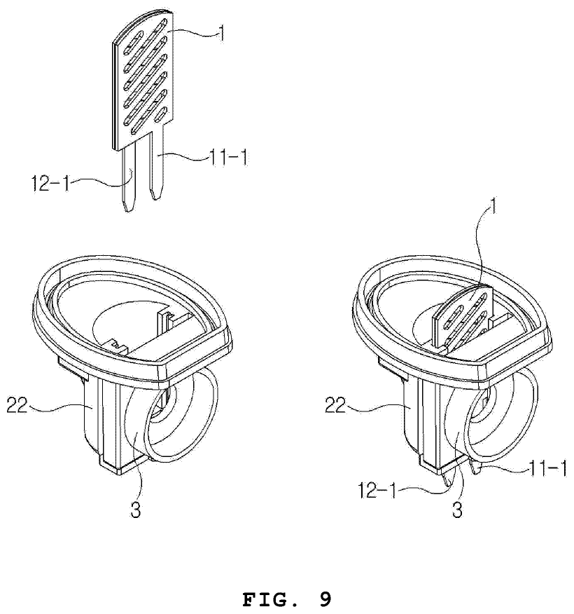

Referring to FIGS. 8 and 9, the lower container 22 has electrode connection terminal through holes 227-1 and 227-2 formed at the lower end of the lower container 22 so that the electrode connection terminals 11-1 and 12-1 of the electrode plates 11 and 12 mounted through the pole plate unit mounting grooves pass through the electrode connection terminal through holes 227-1 and 227-2.

The pole plate unit 1 starts to be inserted into the pole plate unit mounting grooves 224 or 225-1 and 225-2 from an upper end of the lower container 22, and the electrode connection terminals 11-1 and 12-1 are mounted after passing through the electrode connection terminal through holes 227-1 and 227-2 disposed at the lower end of the lower container 22.

The electrode connection terminals 11-1 and 12-1 mounted through the electrode connection terminal through holes 227-1 and 227-2 are connected to the power supply unit 6.

In this instance, the lower container 22 which forms the space for storing raw water together with the upper container 21 is sealed to prevent the raw water from leaking. For this, the electrode connection terminal through holes 227-1 and 227-2 through which the electrode connection terminals 11-1 and 12-1 pass are sealed with a material such as rubber and are waterproofed with a material such as epoxy.

When electric power is supplied to the electrode plates 11 and 12 of the pole plate unit 1, the raw water getting in contact with the electrode plates 11 and 12 is converted into sterilized water.

In order to provide the sterilized water in a mist form using the ultrasonic vibrator 31, the electrode plates 11 and 12 must get in contact with the raw water, and the sterilized water must get in contact with the ultrasonic vibrator 31.

The lower container 22 communicates with the ultrasonic vibrator 31 of the spraying unit 3 through the sterilized water outlet 221. The sterilized water is not inhaled into a suction tub and discharged to the outside, but is naturally supplied to the sterilized water outlet 221 formed in the side of the lower container 22 due to gravity and is supplied to the ultrasonic vibrator 31 of the spraying unit 3.

The spraying unit 3 has the ultrasonic vibrator 31 to make sterilized water into a mist form through the ultrasonic vibrator 31 attached to the sterilized water outlet 221 of the lower container 22 and discharge the mist form sterilized water to the outside.

The ultrasonic vibrator 31 is closely attached to the sterilized water outlet 221 to directly spray the sterilized water from the lower container 22 and to have a close-packed structure. The ultrasonic vibrator 31 has a plurality of spray holes to discharge sterilized water, and may have all forms well-known in this technical field.

As shown in FIG. 1, mist may be discharged in the direction which is vertical to the side of the portable mist device 100 for providing sterilized hydrogen water mist.

The power supply unit 6 supplies electric power to the pole plate unit 1 and the ultrasonic vibrator 31, and may be arranged in various positions. As an example, the power supply unit 6 may be located below the lower container 22.

The power supply unit 6 may be a disposable battery or a rechargeable battery, and may be charged by cable or wirelessly. Moreover, the power supply unit 6 may include an USB terminal capable of being provided with electric power from the outside.

The power supply unit 6 may selectively supply electric power to the pole plate unit 1 and the ultrasonic vibrator 31 to actuate the pole plate unit 1 and the ultrasonic vibrator 31.

The middle and lower housing 7 surrounds the lower container 22 and the power supply unit 6 and may be made of a transparent or opaque material without limit in its form and material. Moreover, the middle and lower housing 7 may be formed integrally with the upper housing 5.

The lid housing 4 may be formed to be combined with the raw water inlet 211 of the upper container 21. The lid housing 4 may serve as a cover or a lid to cover the raw water inlet 211, and may be freely attached to or detached from the raw water inlet 211.

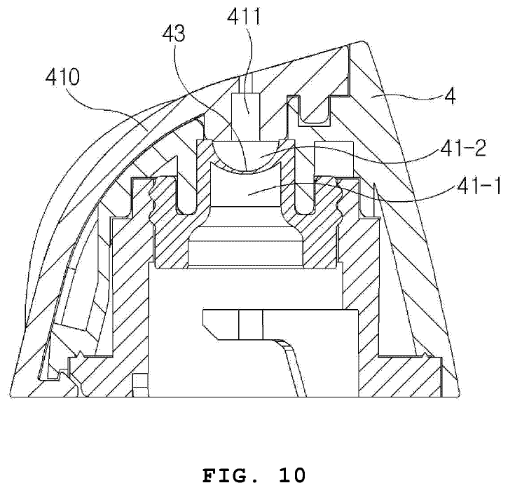

FIG. 10 is a vertically sectional view of the lid housing 4. The lid housing 4 includes gas discharge holes 41-1 and 41-2 for discharging gas ascending from the upper container 21 to the outside. The gas discharge holes 41-1 and 41-2 may be stopped with a member 43 made of a soft material like rubber to prevent leak of the sterilized water.

The gas discharge hole stopping member 43 is disposed to prevent water from being discharged out and to be closed or opened depending on gas pressure.

As an example, the gas discharge hole stopping member 43 may be cut into a predetermined shape, such as a cross shape, at a portion corresponding to the gas discharge hole.

Then, gas pressure rises to open the cross-shaped cut portion, so that the cut portion is opened to discharge gas to the outside.

When gas is discharged properly, the sterilized gas is smoothly discharged through the ultrasonic vibrator 31.

A cover 410 is detachably mounted to the lid housing 4. The cover 410 for covering the lid housing 4 includes a gas discharge hole 411 communicating with the gas discharge hole 41-2 of the lid housing 4.

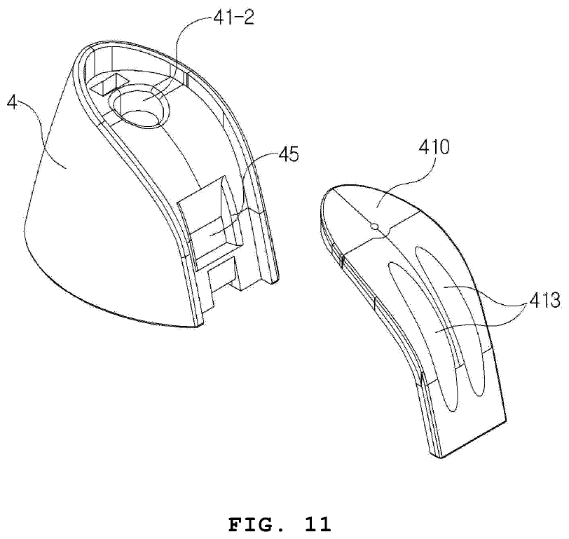

Referring to FIG. 11, the lid housing 4 includes a magnet receiving recess 45 to receive a magnet, and the cover 410 for covering the lid housing 4 includes a massage protrusion 413 for providing massage effect.

A magnetic force can help skin care, and gives a proper stimulus to the skin through a skin massage using the massage protrusion 413 to enhance effect of beauty care. The massage protrusion 413 may be varied in its location, size, number, and shape as occasion demands, and is not limited in its location, size, number, and shape.

As described above, while the present invention has been particularly shown and described with reference to the example embodiments thereof, it will be understood by those of ordinary skill in the art that various changes, modifications and equivalents may be made in the present invention without departing from the technical idea and scope of the present invention.

What is claimed is:

1. A portable mist device for providing sterilized hydrogen water mist comprising:
a sterilized water generating module, which includes an upper container having a raw water inlet, a lower container having a sterilized water outlet formed at the side, and a pole plate unit for generating sterilized water through underwater discharge of the raw water by a pair of electrode plates;
a lid housing disposed to be combined with the raw water inlet;
a spraying unit disposed at the sterilized water outlet to discharge the sterilized water in a mist form to the outside through an ultrasonic vibrator; and
a power supply unit for supplying electric power to the pole plate unit and the ultrasonic vibrator,
wherein the lower container has pole plate unit mounting grooves to detachably mount the pole plate unit, the electrode plates are vertically mounted in the pole plate unit mounting grooves and the pole plate unit mounting grooves are located parallel with the sterilized water outlet.

2. The portable mist device according to claim 1, further comprising:
an upper housing for surrounding the upper container; and
a middle and lower housing for surrounding the lower container and the power supply unit,
wherein the power supply unit is located below the lower container.

3. The portable mist device according to claim 1, wherein the pole plate unit includes a pair of electrode plates and a partition plate arranged between the electrode plates, and
wherein the pole plate unit mounting grooves are formed in such a way that the pole plate unit is detachably mounted.

4. The portable mist device according to claim 1, wherein the lower container has two pole plate unit mounting grooves in which a pair of the electrode plates are individually attached and detached, and
wherein a partition plate is formed between the two grooves to prevent physical contact of the two electrode plates.

5. The portable mist device according to claim 1, wherein each of the electrode plates has an electrode connection terminal extending and protruding in a vertical direction to a predetermined length from a lower end thereof.

6. The portable mist device according to claim 5, wherein the lower container has an electrode connection terminal through hole formed at a lower end thereof so that the electrode connection terminal of each electrode plate mounted through the pole plate unit mounting grooves pass therethrough.

7. The portable mist device according to claim 1, wherein the lid housing has a gas discharge hole for discharging gas to the outside,
wherein the gas discharge hole is stopped with a member made of a soft material, and
wherein the gas discharge hole stopping member is cut in a predetermined shape to be opened and closed depending on gas pressure.

8. The portable mist device according to claim 1, wherein the lid housing has a magnet receiving recess to receive a magnet, and
wherein a cover for covering the lid housing has a massage protrusion formed at a position corresponding to the magnet receiving recess to provide massage effect.
